(19) United States
(12) Patent Application Publication
(10) Pub. No.: US 2016/0313340 A1
(43) Pub. Date: Oct. 27, 2016

(54) IMPROVED VERTICAL FLOW IMMUNOASSAY

(71) Applicant: NORWEGIAN ANTIBODIES AS, As (NO)

(72) Inventor: Truls Johan Biornstad, As (NO)

(21) Appl. No.: 15/101,450

(22) PCT Filed: Dec. 17, 2014
(86) PCT No.: PCT/EP2014/078215

§ 371 Date: Jun. 3, 2016

(30) Foreign Application Priority Data
Dec. 19, 2013 (NO) 20131723

(51) Int. Cl. G01N 33/58

(52) U.S. Cl. CPC G01N 33/585; G01N 2333/4737

(57) ABSTRACT

A vertical flow membrane assay device for quantitative determination of an analyte in a sample comprising at least a porous membrane and binding molecules attached to colored particles suspended in a buffer, wherein said porous membrane has an average pore size of about 2 μm to about 6 μm, said colored particles have an average diameter in the interval of 80 nm to about 400 nm, and said buffer has a pH in the interval of 7.5 to 1 l. A method for performing a vertical flow membrane assay using a device as disclosed, and/or performing the corresponding method steps.

### TECHNICAL FIELD

The present disclosure relates to the field of immunoassays, in particular point-of-care tests, where speed, easy handling and robustness are required features. The disclosure specifically relates to a vertical flow immunoassay employing colored particles conjugated to binding molecules with specific affinity for an analyte of interest.

### BACKGROUND

Immunoassays have been used for several decades, and are today important tools in both human and veterinary medicine and research. Information gained by clinical immunoassay testing has shortened hospital stays and decreased the severity of illness by assisting medical staff in rapidly identifying and assessing the progression of disease, thereby leading to improved therapeutic choices. In life science research, immunoassays are used in the study of biological systems by tracking different proteins, hormones, and antibodies. In industry, immunoassays are used to detect contaminants in food and water, and in quality control to monitor specific molecules used during product processing.

Immunoassays are available in many different configurations, and for many different purposes. The present description concerns in particular immunoassays for assessment of analytes in body liquids like whole blood, blood serum, blood plasma and/or urine, and especially analytes for which specific antibodies or other binding molecules can be made. The use of antibodies is well known to the skilled man of the art, and an overview of antibodies, polyclonal, monoclonal and immunoreactive fragments of antibodies or derivatives of antibodies is presented in “Antibodies: A Laboratory Manual” by Ed Harlow and David P. Lane, ISBN 0-8769-314-2.

One particular type of immunoassay is the flow-through filter based immunoassay system, often called a vertical flow immunoassay. In a vertical flow immunoassay, a sample aliquot or a diluted sample aliquot is passed through a filter membrane on which binding molecules with specific reactivity to an analyte of interest have been immobilized. A suspension of labeled particles with immobilized binding molecules with specific reactivity to the same analyte is then added to the same membrane, and non-bound particles are washed away. When the binding molecules are antibodies, fragments or derivatives thereof, the particles are called immunoparticles. The amount of labeled particles remaining on the membrane can be assessed by measuring a signal associated with the label, for example light reflectance in the case of colored particles.

U.S. Pat. No. 5,096,809 discloses a method for detecting an analyte in a sample of whole blood using a porous membrane support assay device. In one embodiment, the method comprises the steps of providing an assay device comprising a liquid-permeable support layer having on its surface a means for detecting the presence of a selected analyte, applying a sample of whole blood to the layer, drawing the colored components of the blood through the layer, and then visually indicating on the layer the presence or absence of the analyte in the blood sample. In another embodiment of the invention, the method further comprises the step of lysing the red blood cells in the sample to release the colored components within and to permit the colored components to be drawn through the layer. In one variation, the lysing of the red blood cells is performed prior to applying the blood sample to the layer, and in another variation the lysing step is performed after applying the blood sample to the layer. According to U.S. Pat. No. 5,096,809 the membrane may have pores on average no greater than 25 μm in size. In a preferred embodiment, the membrane has pores on average no greater than 10 μm in size, and in a particularly preferred embodiment, the membrane has an average pore size of about 5 μm.

U.S. 2006/0172435 discloses a non-continuous immunoassay device which includes two or more separated pads for immunoassay analysis, and is capable of controlling the migration speed of a mobile phase between the separated pads, and an immunoassay method using the same are disclosed. The immunoassay device includes a first pad receiving a mobile phase; a second pad which is spatially separated from the first pad by a predetermined distance, and to which the mobile phase migrates; an upper case for covering the upper parts of the first pad and the second pad; a lower case for covering the lower parts of the first pad and the second pad; and a connecting member which is formed on at least one of the upper case and the lower case, and located between the first pad and the second pad to form a passage for moving the liquid sample.

U.S. 2005/0196875 relates to a dry reagent assay device having two or more test zones for detecting two or more analytes in a test sample. In one embodiment, the test zones may be the site of a binding reaction between members of a binding pair, or they may be the site of a general chemistry reaction. The disclosure also relates to methods for performing assays to detect analytes in test samples using such devices.

U.S. 2005/0244986 shows an analytical test device useful for example in pregnancy testing, comprising a hollow casing constructed of moisture-impervious solid material, such as plastics materials, containing a dry porous carrier which communicates indirectly with the exterior of the casing via a bibulous sample receiving member which protrudes from the casing such that a liquid test sample can be applied to the receiving member and permeate therefrom to the porous carrier, the carrier containing in a first zone a labelled specific binding reagent is freely mobile within the porous carrier when in the moist state, and in a second zone spatially distinct from the first zone unlabeled specific binding reagent for the same analyte which unlabeled reagent is permanently immobilized on the carrier material and is therefore not mobile in the moist state, the two zones being arranged such that liquid sample applied to the porous carrier can permeate via the first zone into the second zone, and the device incorporating means, such as an aperture in the casing, enabling the extent (if any) to which the labelled reagent becomes bound in the second zone to be observed.

U.S. 2012/0015448 relates to an assay device for determining the presence and/or extent of one or more analytes in liquid sample containing a) first and second assays each comprising a flow-path having a detection zone for immobilizing a labelled binding reagent, wherein detection of a labelled binding reagent at one or both detection zones is indicative of the presence and/or extent of one or more analytes; b) a shared reference zone; c) one or more light sources to illuminate the detection zones and the reference zone; d) one or more photo detectors to detect light from the detection zones and the reference zone, which photo detector/s generate a signal, the magnitude of which signal is related to the amount of light detected; and e) signal processing means for processing signals from the photo detector/s.

WO 2007/052008 teaches an assay method for a cell-containing body sample, said method comprising treating said sample under conditions whereby to cause cell lysis, preferably by means of a detergent; and subjecting the thus-generated lysed sample to conditions causing the cleavage of nucleic acid molecules. The invention additionally provides the use of nucleic acid cleavage conditions in enhancing a membrane assay, a device for carrying out such an assay, and a kit for use in the assay. WO 2007/052008 teaches the use of membranes with a pore size of less than 2 μm, preferably 0.45 μm, detergents and/or enzymes, and calcium and/or magnesium ions to facilitate the effect of enzymes added.

Oh et al., Vertical flow immunoassay (VFA) biosensor for a rapid one-step immunoassay, Lab Chip. 2013 Mar. 7;13(5):768-72, discloses an one-step immunoassay of high sensitivity C-reactive protein (hsCRP) using a biosensor with a vertical flow immunoassay (VFA). The VFA biosensor was primarily composed of a sample pad, conjugate pad, FTH film and nitrocellulose (NC) membrane, which were all vertically stacked upon one another. Anti-hsCRP and secondary antibodies were consecutively immobilized on the NC membrane at the position below the holes. Gold nanoparticles (AuNPs) conjugated with another anti-hsCRP antibody were encapsulated in the conjugation pad. Various assay conditions, including the size of the hole and the sample volume, were optimized. Under optimized conditions, hsCRP concentrations from 0.01 to 10 pg/mL were detected within 2 min. In comparison with a lateral flow assay (LFA) system, the VFA sensor showed a gradual increase of signal in a concentration-dependent manner without a hook effect in the tested range. This test is based on the use of very small (10-40 nm) gold nanoparticles in order to detect very low concentrations of analyte. The test is also adapted for serum samples, and less suitable for whole blood. Further, a detection time of 2 minutes leaves room for improvement. It is also not clear if the disclosed test is quantitative.

### SUMMARY

An object of the present disclosure is to make available an improved vertical flow immunoassay for the handling of samples of body fluids, in particular whole blood, blood serum, blood plasma, and hemolysates of whole blood. Point-of-care use and in particular field testing, both in human and veterinary medicine, requires simple and fast, but yet reliable assays. There is a need for rapid, simple, robust and reliable immune assays for point-of-care tests and in particular field testing in human and veterinary medicine, and in particular for acute phase proteins such as C-reactive protein (CRP), haptoglobin, fibrinogen and/or serum amyloid A protein (SAA) in samples of whole blood.

The embodiments presented herein provide a surprising solution to the challenge of background versus signal problems and false high readings using vertical flow filter-immunoassays based on the use of immunoparticles. The tendency of clogging of the filter because of particulate matter or solid substances, remaining cellular material and whole molecules or fragments of nuclear materials, such as DNA and histones, was overcome without including enzymes and divalent ions in the assay mixture.

One embodiment is a vertical flow membrane assay for quantitative determination of an analyte in a sample, comprising at least a porous membrane and binding molecules attached to colored particles suspended in a buffer, wherein said porous membrane has an average pore size of 2 μm to about 6 μm, said colored particles have an average diameter in the interval of 80 nm to about 400 nm, and said buffer has a pH in the interval of about 7.5 to 11, preferably in the interval of 8.5 to 11.

According to a preferred aspect of this first embodiment, said analyte is chosen from C-reactive protein (CRP), haptoglobin, fibrinogen, and/or serum amyloid A protein (SAA), more preferably said analyte is CRP.

According to a preferred aspect of the first embodiment, freely combinable with the above aspects, the sample is a sample taken from a an animal, for example but not limited to humans, domesticated animals such as pets and utility animals, such as dogs, cats, horses, camels, donkeys, birds, etc., and livestock, such as cattle, pigs, sheep, goats, etc.

According to a preferred aspect of the first embodiment, freely combinable with the above aspects, the average membrane pore size is about 3.0 pm and the average particle size is in the interval of about 100 to about 200 nm.

According to a particularly preferred aspect of the first embodiment, freely combinable with the above aspects, the average membrane pore size is about 3.0 μm and the average particle size is 124 nm.

According to a preferred aspect of the first embodiment, freely combinable with the above aspects, the particles are monodisperse polymeric particles, for example but not limited to monodisperse polymeric particles chosen from plastic particles such as styrene-based polymeric particles, such as polystyrene particles, acrylate-based polymeric particles, and latex particles.

According to a preferred aspect of the first embodiment, freely combinable with the above aspects, said sample is a sample chosen from mammalian whole blood, blood plasma, blood serum, and blood hemolysate.

According to a particularly preferred aspect of the first embodiment, freely combinable with the above aspects, said analyte is canine CRP and said sample is whole blood obtained from a dog.

According to another aspect of the first embodiment, freely combinable with the above aspects, at least one reference spot of colored material is arranged on the assay.

Another embodiment of the present disclosure is a novel method for the quantitative determination of an analyte in a sample, using binding molecules with specific affinity for said analyte attached to colored particles forming immunoparticles, wherein said immunoparticles are suspended in a buffer, comprising the steps of
hypotonically lysing erythrocytes and optionally diluting the sample,
adding the lysed and optionally diluted sample to a membrane,
adding said immunoparticles to said membrane,
adding a wash buffer to said membrane, and
measuring the intensity of the color of the membrane,

wherein said colored particles immunoparticles have an average diameter in the interval of 80 nm to about 400 nm; said porous membrane has as average pore size of about 2 μm to about 6 μm; and said buffer has a pH in the interval of 7.5 to 11, preferably in the interval of 8.5 to 11.

According to a preferred aspect of the method, said analyte is chosen from C-reactive protein (CRP), haptoglobin, fibrinogen, and/or serum amyloid A protein (SAA), more preferably said analyte is CRP.

According to a preferred aspect of the method, freely combinable with the above aspects, said sample is a sample chosen from whole blood, blood plasma, blood serum, and blood hemolysate.

According to a preferred aspect of the method, freely combinable with the above aspects, the sample is a sample taken from a an animal, for example but not limited to humans, domesticated animals such as pets and utility animals, such as dogs, cats, horses, camels, donkeys, birds, etc., and livestock, such as cattle, pigs, sheep, goats, etc.

According to another aspect of the method, freely combinable with the above aspects, the lysed sample is mixed with a suspension of immunoparticles prior to addition to said membrane.

According to a preferred aspect of the method, freely combinable with the above aspects, said analyte is canine CRP and said sample is whole blood obtained from a dog.

According to a preferred aspect of the method, freely combinable with the above aspects, the average membrane pore size is about 3.0 μm and the average particle size is in the interval of about 100 to about 200 nm.

According to a preferred aspect of the method, freely combinable with the above aspects, the average membrane pore size is about 3.0 μm and the average particle size is 124 nm.

According to a preferred aspect of the method, freely combinable with the above aspects, the particles are monodisperse polymeric particles, for example but not limited to monodisperse polymeric particles chosen from plastic particles such as styrene-based polymeric particles, such as polystyrene particles, acrylate-based polymeric particles, and latex particles.

According to a preferred aspect of the method, freely combinable with the above aspects, a reference value is obtained by measuring the intensity of the color of a reference spot arranged on the assay.

### BRIEF DESCRIPTION OF DRAWINGS

The invention is now described, by way of example, with reference to the accompanying drawings, in which:

FIG. 1 schematically shows an embodiment wherein a membrane is arranged in a holder, having an aperture or well for receiving a sample, reagents and wash solution.

FIG. 2 is a photography showing the results from one experiment using one particle size (124 nm) and varying the membrane pore size from 0.1 μm to 12 μm.

FIG. 3 is a graphical representation of measurement of signal for each cCRP test (done individually and uploaded into Adobe Photoshop Elements 13©). The function “Eyedropper Tool” was used to determine the Hue (h) number of the HSB value detected and was used to produce curves of signal of each test.

FIG. 4 is a graphical representation of measurement of background for each cCRP test (done individually and uploaded into Adobe Photoshop Elements 13©). The function “Eyedropper Tool” was used to determine the Hue (h) number of the HSB value detected and was used to produce curves of background of each test.

### DESCRIPTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In addition to the above, the following terms will be used:

The term “immunomembrane” is used to describe a porous membrane which allows liquids and very small particles suspended in a liquid to pass through the membrane, and which has antibodies immobilized to the solid material of the membrane.

The term “immunoparticle” is used to describe particles, preferably labeled or colored particles carrying binding molecules with specific affinity for a desired analyte.

One embodiment of the present disclosure is a vertical flow membrane assay for quantitative determination of an analyte in a sample, comprising at least a porous membrane and binding molecules attached to colored particles suspended in a buffer, wherein said porous membrane has an average pore size of about 2 μm to about 6 μm, said colored particles have an average diameter in the interval of 80 nm to about 400 nm, and said buffer has a pH in the interval of about 7.5 to 11, preferably in the interval of 8.5 to 11.

The porous membrane can be made from nitrocellulose, but numerous other materials for physical absorption or covalent coupling are well known to the person skilled in the art. Examples of membranes include, but are not limited to nitrocellulose membranes, blotting membranes, diethylaminoethyl ion exchange paper and blot adsorbent filter papers. It is also contemplated that any support material can be used, provided that it has functional groups to which an adsorbent molecule (either a receptor or a ligand) can be covalently attached. Examples of such materials include aminobenzyl-oxymethyl (ABM) paper, 2-aminophenyl thioether (APT) paper, cyanogen bromide activated paper (CBA) (see Methods in Enzymology, R. Wu (ed.) 1979, Academic Press New York, 68:436-442 for a discussion of CBA paper), diazobenzyloxymethyl cellulose paper (DBM), diazophenylthioether cellulose paper (DPT) and nitrobenzyloxymethyl cellulose paper (NBM).

Reagents can be coupled to the support using different methods known to a person skilled in the art. The choice of coupling method depends on the chemical composition of the support and the chemical composition of the chemical to be coupled to the support. Chemicals can be coupled to a support by use of cyanogen bromide coupling, silation, diazo coupling, carbodiimide coupling, glutaraldehyde coupling and the use of heterobifunctional reagents.

The immunoparticles are made from preferably labeled or colored particles by coupling binding molecules having specific affinity for a desired analyte, to said particles. The particles are preferably monodisperse polymeric particles, for example but not limited to monodisperse polymeric particles chosen from plastic particles such as styrene-based polymeric particles, such as polystyrene particles, acrylate-based polymeric particles, and latex particles.

Preferably an immunomembrane made from nitrocellulose or another membrane suitable for immobilizing antibodies or other binding molecules or fragments thereof, is used. Preferably the membrane has an average pore size of about 2 μm to about 6 μm, and said colored particles have an average diameter in the interval of 80 nm to about 400 nm. More preferably the membrane has an average pore size of about 3 μm, and the colored particles have an average diameter of about 100-150 nm, most preferably about 124 nm.

According to a preferred aspect of both the device and the method, freely combinable with the above mentioned aspects, the membrane has a detection surface, onto which the sample and reagents is/are applied, wherein the area of said detection surface is significantly smaller than the total area of the membrane and/or the absorption pad placed under the membrane. The ratio of area of the detection surface to the area of the absorption pad is at least 1:2, preferably at least 1:4 and more preferably at least 1:8.

This ratio can also be expressed as the capacity of the absorption pad in relation to the total volume of sample and reagents to be added. Preferably the absorption pad has the capacity to absorb more than 100% of the total volume of sample and reagents to be added, preferably more than 150%, more preferably about 200% or more of the total volume of sample and reagents to be added.

According to a preferred aspect of the first embodiment, said analyte is chosen from C-reactive protein (CRP), haptoglobin, fibrinogen, and/or serum amyloid A protein (SAA), more preferably said analyte is CRP.

According to a preferred aspect of the first embodiment, freely combinable with the above aspects, the average pore size is about 3.0 μm and the membrane average particle size is in the interval of about 100 to about 200 nm.

According to a particularly preferred aspect of the first embodiment, freely combinable with the above aspects, the average membrane pore size is about 3.0 μm and the average particle size is 124 nm. According to a specific embodiment, the membrane is placed on an absorbing pad, and the detection zone on said membrane has a diameter in the interval of 3 to 10 mm, whereas the absorption pad has a diameter which is at least double that of the detection zone.

According to a preferred aspect of the first embodiment, freely combinable with the above aspects, said sample is a sample chosen from whole blood, blood plasma, blood serum, and blood hemolysate.

According to a preferred aspect of the first embodiment, freely combinable with the above aspects, the sample is a sample taken from a an animal, for example but not limited to humans, domesticated animals such as pets and utility animals, such as dogs, cats, horses, camels, donkeys, birds, etc., and livestock, such as cattle, pigs, sheep, goats, etc.

According to a particularly preferred aspect of the first embodiment, freely combinable with the above aspects, said analyte is canine CRP and said sample is whole blood obtained from a dog.

According to another aspect of the first embodiment, freely combinable with the above aspects, at least one reference spot of colored material is arranged on the assay.

There is a direct relationship between the amount of analyte molecules and the color to be measured, since the amount of colored particles bound relates to the amount of analyte molecules present in the sample to be tested. This color is then detectable either visually with comparison to pre-evaluated, pre-calibrated and/or predetermined coloristic diagrams or by measurement of the amount of color by electronic color detectors either freely available on the marked or a specific device developed for the present invention.

A suitable instrument is easily calibrated and adjusted to the immunoparticles used, their color scheme and the desired detection range. A person skilled in the art is familiar with the calibration of detection instruments, for example by using a known amount of analyte, giving a good ratio of background vs. signal, which allows the users to obtain exact readouts. Such systems and methods are described for example in EP 0953149, incorporated herein by reference.

In a special embodiment of the present invention, one or more reference spots are placed or fastened in close proximity to the membrane with immobilized antibodies or bother binding molecules or fragments thereof, preferentially on the holder of the assay membrane. When measuring the signal (e.g. reflectance) from the membrane, this or these reference spots are measured as well. The reading obtained at said at least one reference spot can be used to compensate for instrument-to-instrument and other hardware variations, and to increase the overall accuracy of the assay.

Another embodiment of the present disclosure is a novel method for the quantitative determination of an analyte in a sample, using binding molecules with specific affinity for said analyte attached to colored particles forming immunoparticles, wherein said immunoparticles are suspended in a buffer, comprising the steps of
hypotonically lysing erythrocytes and optionally diluting the sample,
adding the lysed and optionally diluted sample to a membrane,
adding said immunoparticles to said membrane,
passing a wash buffer through said membrane, and
measuring the intensity of the color of the membrane,
   wherein said colored particles have an average diameter in the interval of 80 nm to about 400 nm; said porous membrane has an average pore size of about 2 μm to about 6 μm; and said buffer has a pH in the interval of about 7.5 to 11, preferably in the interval of 8.5 to 11.

According to a preferred aspect of the method, the membrane has a detection zone or detection surface, onto which the sample and reagents is/are applied, wherein the area of said detection zone is significantly smaller than the total area of the membrane and/or the absorption pad placed under the membrane. The ratio of area of the detection surface to the area of the absorption pad is at least 1:2, preferably at least 1:4 and more preferably at least 1:8.

This ratio can also be expressed as the capacity of the absorption pad in relation to the total volume of sample and reagents to be added. Preferably the absorption pad has the capacity to absorb more than 100% of the total volume of sample and reagents to be added, preferably more than 150%, more preferably about 200% or more of the total volume of sample and reagents to be added.

One example of a hypotonic lysis buffer is an aqueous solution comprising 0.5 mg porcine albumin (PSA) per ml, and a small amount preservative to avoid microbiological growth, for example about 0.1% ProClin 950 (Sigma-Aldrich).

One example of a wash buffer is a 30 mM borate buffer having a pH in the interval of 9.2 to 9.3, optionally also comprising about 450 mM sodium chloride, about 10% Tween 20 and about 0.1% ProClin 950 (Sigma-Aldrich).

One example of a particle buffer is a 30 mM borate buffer having a pH in the interval of 9.2 to 9.3, optionally also comprising about 150 mM sodium chloride, about 0.1% Tween 20, about 0.5 mg/ml porcine albumin (PSA) and about 0.1% ProClin 950 (Sigma-Aldrich).

According to a preferred aspect of the method, said analyte is chosen from C-reactive protein (CRP), haptoglobin, fibrinogen, and/or serum amyloid A protein (SAA), more preferably said analyte is CRP.

According to a preferred aspect of the method, freely combinable with the above aspects, said sample is a sample chosen from whole blood, blood plasma, blood serum, and blood hemolysate.

According to a preferred aspect of the method, freely combinable with the above aspects, the sample is a sample taken from a an animal, for example but not limited to humans, domesticated animals such as pets and utility animals, such as dogs, cats, horses, camels, donkeys, birds, etc., and livestock, such as cattle, pigs, sheep, goats, etc.

According to another aspect of the method, freely combinable with the above aspects, the lysed sample is mixed with a suspension of immunoparticles prior to addition to said membrane.

According to a preferred aspect of the method, freely combinable with the above aspects, said analyte is canine CRP and said sample is whole blood obtained from a dog.

According to a preferred aspect of the method, freely combinable with the above aspects, the average pore size is about 3.0 μm and the membrane average particle size is in the interval of about 100 to about 200 nm.

According to a preferred aspect of the method, freely combinable with the above aspects, the average membrane pore size is about 3.0 μm and the average particle size is 124 nm.

According to a preferred aspect of the method, freely combinable with the above aspects, a reference value is obtained by measuring the intensity of the color of a reference spot arranged on the assay. The reference spot can be manufactured separately or integrally with the assay device, using a known concentration of the same label used on the labeled immunoparticles. Techniques for providing a reference spot on an immunometric assay are known to persons skilled in the art.

According to a preferred embodiment of the present disclosure, applying both to the assay and method, the result, i.e. the color intensity is possible to read or measure still within several minutes after the test having been performed. Drying of the test device and the membranes composed—and the microfluidic flow caused by storage and by drying—may however change the color intensity on the test surface. The stability of colored particles bound to analyte molecules in the immunomembrane is sensitive, dependent on blood reflux into the detection membrane, temperature, humidity and/or time elapsed since application of any of the three reagents. The test is therefore dependent on addition of all three reagents in a consecutive order and in a timely fashion, i.e. the application of all three reagents within a 3 minute timeframe. After the test has been run, the time until the test is no longer available for measurement, under normal conditions (10-30° C., RT 60%-90%) is on average 0-30 minutes. This time frame is adequate for user to be able to manually and/or electronically evaluate or measure the color intensity.

An advantage of the present embodiments is the absence of enzymes. First, the use of enzymes in the test reagents constitutes a storage stability challenge. Second, including enzymes in the reagents also increases the price of the products, and also increases the burden of stability testing and control of the enzyme activity in the product. It is important that the accuracy of the test is maintained throughout the time the products are being stored before use.

Further advantages include that the test can be performed using small volumes of sample, e.g. about 10 to about 50 μl. The test can be performed using a whole blood sample, without the need for extracting serum or plasma. The test exhibits an improved signal to background ratio, and lower coefficient of variation (CV).

The test is also very fast, and makes it possible to perform the analysis and reach a result of diagnostic value, in the clinic, at the bedside, in the home, or in the field. The test is also simple and cheap to manufacture, and it can be used without the need for expensive, auxiliary equipment, such as electronic readers etc.

The relationship between the area of the detection zone and the area or rather absorption capacity of the absorbing pad offers a particular advantage in that all the sample and liquid reagents are drawn through the membrane, leaving only the immunoparticles bound to the membrane. This results in an improved signal versus background ratio, and it reduces or eliminates problems with back-flow of sample or reagents, which makes it possible to read the result of the assay within a prolonged period, for example up to 30 minutes after performing the assay. This in turn makes it possible to perform several assays at the same time.

### EXAMPLES

### Example 1

### Preparation of Test Membranes

The following nitrocellulose membranes were tested: 0.1 μm; 0.2 μm; 0.45 μm; 1.0 μm; 3.0 μm; 5.0 μm; 8.0 μm; and 12.0 μm (all from Whatman, GE Healthcare Life Sciences, Norway).

The membrane samples were cut into pieces of equal size, approximately 14 mm×14 mm, suitable for fitting into a prototype assay, here a plastic holder with an absorbent pad underneath.

Each membrane was incubated in a solution of chicken anti-canine CRP antibodies (Norwegian Antibodies AS, Aas, Norway) or immunofragments thereof, typically 3 mg/ml dissolved in 10 mM borate buffer at pH 9.2 to 9.3 by shaking the membrane in the solution at room temperature overnight.

Optionally, the antibodies can be immobilized in the membrane in presence of other proteins or other materials as well, often called carrier proteins or carrier materials. To improve stability of the coated membrane, the membrane was dried at 37° C. for 60 minutes.

Thereafter, the membrane was washed with 3 mg/ml blocking agent, typically egg albumin 3 mg/ml in 30 mM borate buffer at pH 9.2-9.3 with 0.1% Tween 20 and 5 mg/ml porcine serum albumin and 10 mg unspecific antibodies per ml over night at room temperature, before it was dried at room temperature. The membranes were cut and placed in the plastic holders, with an absorbent pad underneath.

Such plastic holders are schematically shown in FIG. 1, both in a cut-out view from above, and a cross section along line D-D. The basic components include a cover (1), a sample addition well (2), defining a detection zone on a membrane, a membrane (3), and an absorbent pad (4). The detection zone has an area, and the absorption pad an area and a volume, the relationship between the areas or the volume, such that the total volume of sample and reagents added to the detection zone are absorbed, and back-flow avoided. In the experimental set up, the absorbent pad had a size of approximately 14 mm×14 mm, whereas the detection zone had a diameter of approximately 5.5 mm. Thus, in that set up, the area of the detection zone was approximately 24 mm² and the area of the absorption pad approximately 196 mm², giving a ratio of about 1:8.

The cover and/or the holder can additionally include markings, instructions for use, a reference spot, a trade mark, a serial number, a bar code, a QR code, RFID-tag etc. (not shown).

### Example 2

### Preparation of Immunoparticles

The following particle sizes were tested: 85 nm; 124 nm; 200 nm; 342 nm; and 506 nm, all ESTAPOR® microspheres (Merck Millipore, Merck KGaA, Darmstadt, Germany).

A solution of chicken anti-canine-CRP (Norwegian Antibodies AS, Lot 24-148-0822), 104 mg IgY in 20 ml final solution was prepared for each batch of labeled immunoparticles, and added to MERCK-ESTAPOR blue latex particles as shown in the table below:

TABLE 1
Particles tested

| Particle size | |
|---|---|
| 85 nm | 0.91 ml of lot 815 |
| 124 nm | 1.33 ml of lot 90-2-A |
| 200 nm | 2.14 ml of lot 697 |
| 342 nm | 3.67 ml of lot 2079/3/2 |
| 506 nm | 5.43 ml of lot 799 |

According to a preferred embodiment blue carboxylated latex particles ESTAPOR®, Merck Millipore), with a mean diameter of 124 nm, are employed. Chicken anti-canine C-reactive protein antibodies (Norwegian Antibodies AS, Lot 24-148-0822) are dialyzed to 40 ml pH 9.5 in 5 mM borate buffer, about 7.5 mM sodium chloride buffer. Said carboxylated blue latex particles are then washed by centrifugation and suspended in 20 ml water. 50 mg EDC (Sigma-Aldrich, US) is dissolved into the particle suspension and the antibody solution is added to the latex suspension, and stirred, e.g. for 5 hours. The particles in the suspension are then washed 4 times with 1 mM sodium chloride, 0.5 mM sodium borate, 0.025 Tween 20, 0.5 mM glycin, pH 9.5. This stock solution is then diluted in a particle buffer. The size of the particles will have an effect on the need of reagents and amount of antibodies used in this protocol and must be adjusted to the square area of surface of particles present in solution.

### Example 3

### Investigation of Signal and Background Using Different Pore Sizes and Particle Sizes

A study was performed to determine levels of signal and levels of background of a cCRP POC test according to embodiments disclosed herein. The results were evaluated both visually, and electronically. In the visual evaluation, the color intensity and background were assessed visually and given a % score, ranging from no signal/no background (white, 0%), to very strong signal/very strong background (dark blue, 100%). In the electronic evaluation, digital pictures were taken and uploaded into Adobe Photoshop. This method allowed graphic presentation of the results. This method also allowed the determination of when the signal is strongest versus when the background is lowest.

The software used throughout entire study of reagents and platform is Adobe Photoshop Elements 13© and the program “Eyedropper tool” to determine HSB and RGB scheme to determine color of uploaded images of all cCRP POC tests.

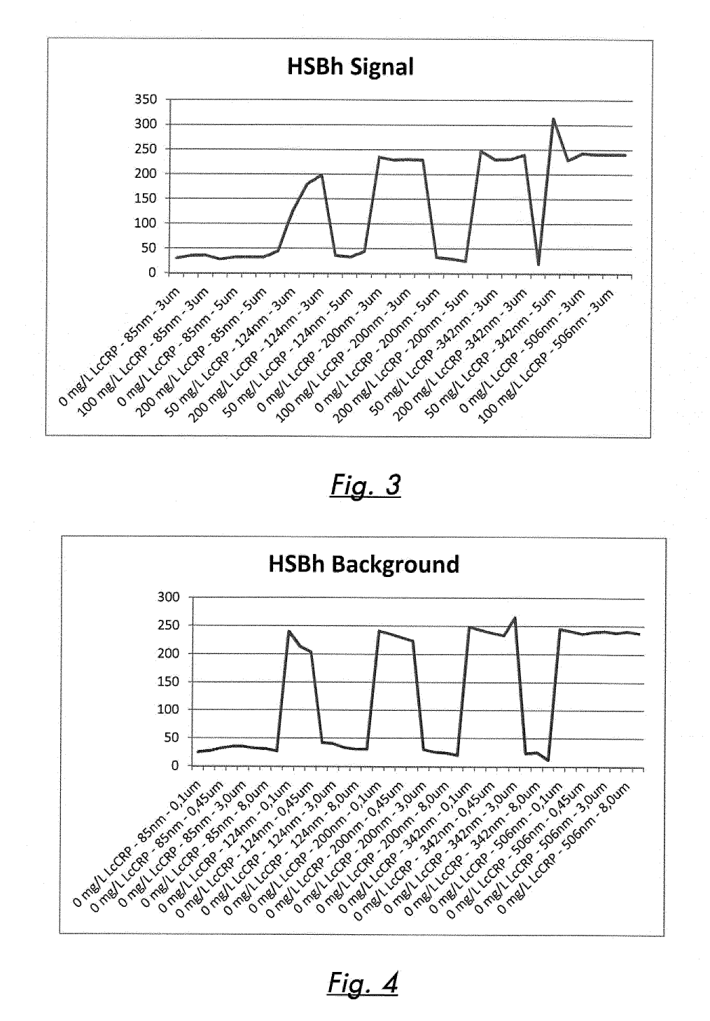

The measurement of each cCRP test was done individually and uploaded into Adobe Photoshop Elements 13©. The HSB scheme provides a device-independent way to describe color. The Hue (h) element of the HSB value was used to produce graphs of both the signal and background of each test (see FIG. 3 and FIG. 4)

Before evaluating the results, a set of features and criteria were set up. These are noted in Table 2 below. At each point in time, during the experiments, these features were detected, measured and it was noted if they fulfilled the required criteria.

TABLE 2
Criteria for evaluating the assays

| Feature | Criteria |
|---|---|
| Color | stable, without any color |
| | released from particles or |
| | added to clear reagents/platform |
| Signal | visible binding of |
| | immunoparticle to reagent on |
| | nitrocellulose membrane |
| Solubility | visible aggregations or |
| | precipitations of reagents or |
| | platform |
| Agglutination (defined as visible | within determined coefficient |
| unspecific clumping of | of variation (CV) |
| immunoparticles compared to baseline | |
| titrated measurements) | |
| General appearance (defined as visible | within determined CV |
| appearance outside of the norm and | |
| compared to baseline determination of | |
| successful platform appearance) | |

The tests were performed in triplicate, and following the protocol presented below:
Hemolysis: 450 μl of buffer was mixed for 10 seconds with 50 μl whole blood sample—total volume 500 μl.
Addition of sample: A sample of 100 μl (hemolysis buffer +blood sample) was added to the membranes prepared in Example 1.
Addition of immunoparticles: 100 μl of the immunoparticles solution prepared in Example 2 was added to the membrane.
Washing step: 100 μl of the wash buffer was added to the membrane.
Application time: 30 to 60 seconds.
Reading time: 1 to 3 minutes, the required stability of output or reading time was however set at 20-30 minutes, allowing for a delay in reading the results.

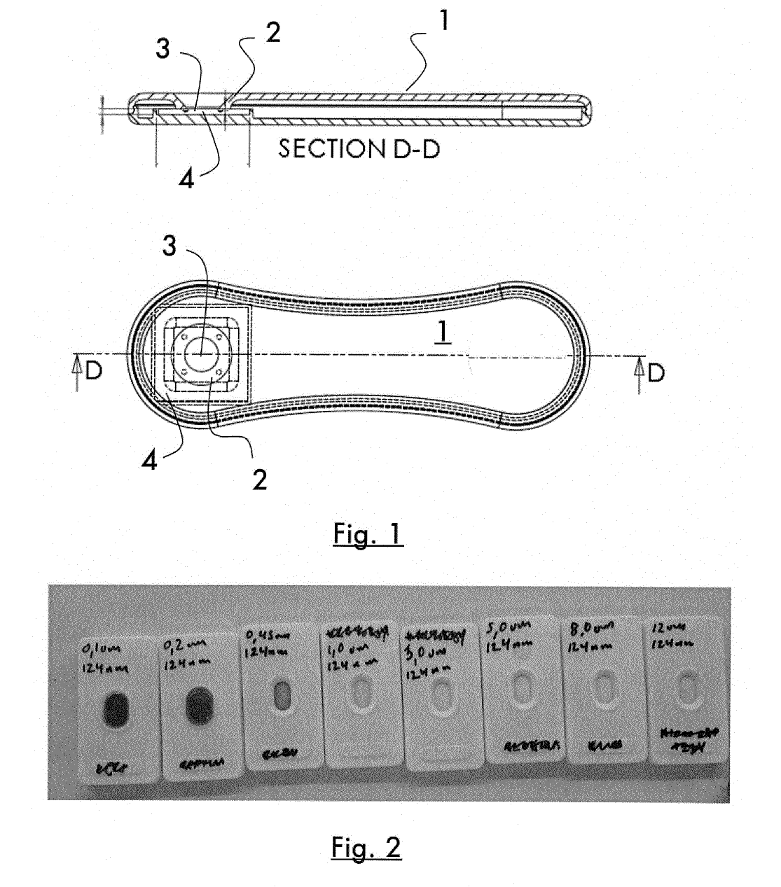

The signal was determined in percentage (0-100%) in a scale from white to dark blue. 0% was determined as original color (white) of nitrocellulose membrane without liquid additive. 100% was determined as dark blue, darker than blue latex immunoparticles in solution. An example of the results is shown in FIG. 2 where eight different membrane average pore sizes are tested using the same average particle size, 124 nm.

Interestingly, the study shows that the test can be conducted and read entirely visually, with fully satisfactory results. The test also shows that an easily available software package can be used to electronically measure the results and determine signal and/or background. The test as such does not require any auxiliary equipment, but it can be validated using freely available software for determination and confirmation of validity, stability and accuracy of the test platform. These results are presented in FIGS. 3 and 4.

The study showed that for the cCRP Point-of-Care assay used as an example in this description, an optimal cross point between signal and background is when the nitrocellulose membrane has a pore size of 3.0 μm and the immunoparticles have a size of 124 nm. This pore size is much larger than expected, but surprisingly and advantageously makes it possible both to immobilize antibodies, and to allow the immobilized antibodies to capture analyte, while simultaneously allowing debris from the sample (whole blood) to flow through without influencing the results.

Similarly, the chosen particle size is larger than expected, in particular when compared to gold particles usually used. The particles were also unexpectedly very soluble and flowed freely through the membrane, while only immunoparticles which had bound to analyte to stay fixed. The immunoparticles were surprisingly able to flow freely through the nitrocellulose membrane even when containing proteins with a higher amount of hydrophobic residues, such as antibodies. Further, the solubility of the immunoparticles in ionized-water made it possible to run the test at a pH which was higher than normal (pH 7.5-11) and higher that what has hitherto been considered to be optimal.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1. A vertical flow membrane assay for quantitative determination of an analyte in a sample chosen from whole blood, blood plasma, blood serum, and blood hemolysate, comprising at least a porous membrane and binding molecules attached to colored particles suspended in a buffer, wherein said porous membrane has an average pore size of about 3.0 μm, said colored particles have an average diameter in the interval of 100 nm to about 200 nm, and said buffer has a pH in the interval of 8.5 to 11.

2. (canceled)

3. The assay according to claim 1, wherein said analyte is chosen from C-reactive protein (CRP), haptoglobin, fibrinogen, and/or serum amyloid A protein (SAA).

4. The assay according to claim 1, wherein said analyte is C-reactive protein (CRP).

5-7. (canceled)

8. The assay according to claim 1, wherein said analyte is canine C-reactive protein (CRP) and said sample is whole blood obtained from a dog.

9. (canceled)

10. The assay according to claim 1, wherein at least one reference spot of colored material is arranged on the assay.

11. A method for the quantitative determination of an analyte in a sample chosen from whole blood, blood plasma, blood serum, and blood hemolysate, using binding molecules with specific affinity for said analyte attached to colored particles forming immunoparticles, wherein said immunoparticles are suspended in a buffer, comprising the steps of:
lysing and optionally diluting the sample,
adding the lysed and optionally diluted sample to a membrane,
adding said immunoparticles to said membrane,
adding a wash buffer to said membrane, and
measuring the intensity of the color of the membrane,
wherein said colored particles have an average diameter in the interval of 80 nm to about 400 nm; said porous membrane has an average pore size of about 2 μm to about 6 μm, and said buffer has a pH in the interval of 7.5 to 11.

12. The method according to claim 11, wherein said buffer has a pH in the interval of 8.5 toll.

13. The method according to claim 11, wherein said analyte is chosen from C-reactive protein (CRP), haptoglobin, fibrinogen, and/or serum amyloid A protein (SAA).

14. The method according to claim 11, wherein said analyte is C-reactive protein (CRP).

15. (canceled)

16. The method according to claim 11, wherein the lysed sample is mixed with a suspension of immunoparticles prior to addition to said membrane.

17. The method according to claim 11, wherein said analyte is canine CRP and said sample is whole blood obtained from a dog.

18. The method according to claim 11, wherein the average membrane pore size is about 3.0 μm, the colored particles are colored polymeric particles, and the average particle size is in the interval of about 100 to about 200 nm.

19. The method according to claim 11, wherein the average membrane pore size is about 3.0 μm and the average particle size is 124 nm.

20. (canceled)

21. The method according to claim 11, wherein a reference value is obtained by measuring the intensity of the color of a reference spot arranged on the assay.
